(19) United States
(12) Patent Application Publication
(10) Pub. No.: US 2023/0071518 A1
(43) Pub. Date: Mar. 9, 2023

(54) INTRANASAL mRNA VACCINES

(71) Applicant: eTheRNA immunotherapies NV, Niel (BE)

(72) Inventors: Wim TIEST, Niel (BE); Diane VAN HOORICK, Niel (BE)

(21) Appl. No.: 17/799,118

(22) PCT Filed: Feb. 15, 2021
(86) PCT No.: PCT/EP2021/053633

§ 371 Date: Aug. 11, 2022

(30) Foreign Application Priority Data
Feb. 14, 2020 (EP) 20157300.3

(51) Int. Cl. A61K 39/12; A61K 39/00; A61P 31/00; A61K 9/00

(52) U.S. Cl. CPC A61K 39/12; A61K 39/001138; A61K 39/001129; A61P 31/00; A61K 9/0043; A61K 2039/5156; A61K 2039/53; A61K 2039/543

(57) ABSTRACT

The present invention in general to intranasal mRNA vaccines, more in particular comprising one or more immunostimulatory molecules, one or more pathogenic antigens and a specifically designed delivery system. Specifically said immunostimulatory molecules and pathogenic antigens are provided for in the form of mRNA molecules encoding such molecules and antigen; more in particular mRNA molecules encoding for CD40L, caTLR4 and/or CD70 in combination with one or more mRNA molecules encoding a bacterial, viral or fungal antigen. Specifically said, the delivery is a mixture of chemical compounds that allow protection and deposition of the vaccine and targeting to the antigen presenting cells in the nose. In particular, present invention is well suited for development of a rapid response vaccine in an outbreak setting.

### FIELD OF THE INVENTION

The present invention in general to intranasal mRNA vaccines, more in particular comprising one or more immunostimulatory molecules, one or more pathogenic antigens and a specifically designed delivery system. Specifically said immunostimulatory molecules and pathogenic antigens are provided for in the form of mRNA molecules encoding such molecules and antigen; more in particular mRNA molecules encoding for CD40L, caTLR4 and/or CD70 in combination with one or more mRNA molecules encoding a bacterial, viral or fungal antigen. Specifically said, the delivery is a mixture of chemical compounds that allow protection and deposition of the vaccine and targeting to the antigen presenting cells in the nose. In particular, present invention is well suited for development of a rapid response vaccine in an outbreak setting.

### BACKGROUND TO THE INVENTION

Past efforts around vaccine for outbreak infectious diseases like SARS and MERS have had limited impact because the vaccines transpired after the epidemic peak, and the technology used did not allow broader coverage and re-use in subsequent outbreaks. Current efforts for COVID-19 (nCoV-2019) vaccine design capitalize on technologies that induce high levels of systemic neutralizing antibodies. Antibody responses in patients recovering from SARS or MERS infection were however reported to be short-lived in nature and of limited cross-reactivity against related strains. In contrast, T cell responses to Coronaviruses appear long-lived and of significant cross-reactivity.

Mucosal, and in particular intranasal, T cell immunity is advanced as a key tool in preventing lower respiratory tract infection and disease for several airborne viral pathogens. Intranasal administration of mRNA has been shown in mice under very specific circumstances to induce such strong immunity. The use of T cell immunity as primary defense makes the approach more robust against known variability in the viral proteins targeted by humoral immune responses, and sets hopes for protection against strain drift and even future Corona variants. Intranasal vaccination with mRNA has the potential to induce such mucosal T cell responses. Moreover, intranasal delivery is a proven vaccine technology with FluMist® on the market.

TriMix, a mix of thee mRNAs encoding the immunostimulatory proteins CD40L, CD70 and a constitutively active form of TLR4 (caTLR4) has been demonstrated to enhance the magnitude and quality of T cell responses against co-delivered mRNA encoded antigens in the context of therapeutic cancer vaccines upon intradermal, intravenous and intranodal mRNA vaccine administration. Here, we demonstrate that co-administration of TriMix mRNA with antigen encoding mRNA can enhance the efficacy of intranasal vaccination against respiratory viruses. Human coronaviruses (HCoVs) have long been considered inconsequential pathogens, causing the “common cold” in otherwise healthy people. However, in the 21st century, 2 highly pathogenic HCoVs—severe acute respiratory syndrome coronavirus (SARS-CoV), Middle East respiratory syndrome coronavirus (MERS-CoV)—emerged from animal reservoirs to cause global epidemics with alarming morbidity and mortality.

Coronaviruses are enveloped RNA viruses that are distributed broadly among humans, other mammals, and birds and that cause respiratory, enteric, hepatic, and neurologic diseases. Six coronavirus species are known to cause human disease. Four viruses—229E, OC43, NL63, and HKU1—are prevalent and typically cause common cold symptoms in immunocompetent individuals. The two other strains—severe acute respiratory syndrome coronavirus (SARS-CoV) and Middle East respiratory syndrome coronavirus (MERS-CoV)—are zoonotic in origin and have been linked to sometimes fatal illness. SARS-CoV was the causal agent of the severe acute respiratory syndrome outbreaks in 2002 and 2003 in Guangdong Province, China. MERS-CoV was the pathogen responsible for severe respiratory disease outbreaks in 2012 in the Middle East.

Common symptoms of SARS included fever, cough, dyspnea, and occasionally watery diarrhea. Of the infected patients, 20% to 30% required mechanical ventilation and 10% died, with higher fatality rates in older patients and those with medical comorbidities. Human-to-human transmission was documented, mostly in health care settings. This nosocomial spread may be explained by basic virology: the predominant human receptor for the SARS S glycoprotein, human angiotensin-converting enzyme 2 (ACE2), is found primarily in the lower respiratory tract, rather than in the upper airway. Receptor distribution may account for both the dearth of upper respiratory tract symptoms and the finding that peak viral shedding occurred late (≈10 days) in illness when individuals were already hospitalized. SARS care often necessitated aerosol-generating procedures such as intubation, which also may have contributed to the prominent nosocomial spread.

MERS shares many clinical features with SARS such as severe atypical pneumonia, yet key differences are evident. Patients with MERS have prominent gastrointestinal symptoms and often acute kidney failure, likely explained by the binding of the MERS-CoV S glycoprotein to dipeptidyl peptidase 4 (DPP4), which is present in the lower airways as well as kidney and gastrointestinal tract. MERS necessitates mechanical ventilation in 50% to 89% of patients and has a case fatality rate of 36%.

In December 2019, a cluster of patients with pneumonia of unknown cause was linked to a seafood wholesale market in Wuhan, China. A previously unknown betacoronavirus was discovered through the use of unbiased sequencing in samples from patients with pneumonia. Human airway epithelial cells were used to isolate a novel coronavirus, named COVID-19, which formed another clade within the subgenus sarbecovirus, orthocoronavirinae subfamily. Different from both MERS-CoV and SARS-CoV, COVID-19 is the seventh member of the family of coronaviruses that infect humans.

No human vaccines against Coronavirus are registered or even further than phase I development. There do exists a number of (life-attenuated) veterinary corona vaccines (canine, feline).

At each outbreak an accelerated vaccine development was kicked off. The length of development however makes that incidence (and thus the possibility to test vaccine efficacy) has already dropped to low levels by the time a vaccine candidate makes it past phase I. Subsequent outbreaks are of a different viral subtype, and so previous effort cannot be used.

Based on the SARS outbreak a number of US, EU and Asian vaccine developers moved candidates through preclinical development, and a few were actually tested in phase I. (Roper & Rehm, 2009) Being 2003, vaccine technology employed includes several live attenuated viruses, a few subunit vaccines, some adeno-based and some DNA vaccines.

Data learn that induction of a strong systemic antibody response (eg against spike protein) is not a guarantee for neutralization. Roper et al, 2009 pose that intranasal vaccination may well be the route of choice for prevention of transmission by inducing strong IgA responses.

To provide an answer to these lengthy development processes of novel vaccines at the time of outbreak of respiratory diseases, we have now developed a novel vaccine platform comprising: one or more mRNA molecules encoding for a functional immunostimulatory protein selected from the list comprising CD40L, caTLR4 and CD70; and one or more mRNA molecules encoding a bacterial, viral or fungal antigen; in the form of an intranasal formulation. Such platform approach is highly suitable for rapid development of vaccines at the time of outbreak of novel or even existing respiratory pathogens.

### SUMMARY OF THE INVENTION

In a first aspect, the present invention provides a combination comprising:
one or more mRNA molecules encoding for a functional immunostimulatory protein selected from the list comprising CD40L, caTLR4 and CD70; and
one or more mRNA molecules encoding a bacterial, viral or fungal antigen or an artificial antigen designed to contains T cell stimulatory epitopes and suppress T regulatory epitopes.

wherein said combination is in the form of an intranasal formulation.

In a specific embodiment, said one or more mRNA molecules encode for all of said functional immunostimulatory proteins CD40L, caTLR4 and CD70.

In yet a further embodiment, said antigen is an antigen from a respiratory tract pathogen, such as a coronavirus.

In another particular embodiment, said antigen is M (matrix), N (nucleocapid) S (spike) antigen or a virus-encoded non-structural protein (NSP); in particular M (matrix), N (nucleocapid) S (spike) antigen.

In another particular embodiment, said antigen is an artificially composed immunogen composed of several epitopes from the pathogen's genome.

In yet a further embodiment of the present invention, said mRNA molecules are formulated in the form of lipid or polymer based nanoparticles, including lipid-based nanoparticles, or a dendrimer, polyplex, lipoplex, hybrid lipopolyplex or polylipoplex formulation; such as lipid-based nanoparticles or a lipoplex or polylipoplex formulation.

In a further aspect the present invention also provides a vaccine comprising a combination as defined herein.

The whole invention comprises the combination with an appropriate delivery device and use protocol that maximizes delivery and exposure to the nose and minimizes lung exposure.

In addition, the present invention provides the combination or vaccine as defined herein for use in human or veterinary medicine; specifically for use in the prevention and/or treatment of an infectious disease.

### DETAILED DESCRIPTION OF THE INVENTION

As already detailed herein above, the present invention provides a combination comprising:
one or more mRNA molecules encoding for a functional immunostimulatory protein selected from the list comprising CD40L, caTLR4 and CD70; and
one or more mRNA molecules encoding a bacterial, viral or fungal antigen, in particular mRNA molecules designed for induction of antibody response; or alternatively an artificial antigen designed to contains T cell stimulatory epitopes and suppress T regulatory epitopes;

wherein said combination is in the form of an intranasal formulation.

In a specific embodiment, said combination comprises TriMix, i.e. mRNA molecules encoding all of said CD40L, caTLR4 and CD70 immunostimulatory proteins.

Throughout the invention, the term “TriMix” stands for a mixture of mRNA molecules encoding CD40L, CD70 and caTLR4 immunostimulatory proteins. The use of the combination of CD40L and caTLR4 generates mature, cytokine/chemokine secreting DCs, as has been shown for CD40 and TLR4 ligation through addition of soluble CD40L and LPS. The introduction of CD70 into the DCs provides a co-stimulatory signal to CD27⁺ naive T-cells by inhibiting activated T-cell apoptosis and by supporting T-cell proliferation. As an alternative to caTLR4, other Toll-Like Receptors (TLR) could be used. For each TLR, a constitutive active form is known, and could possibly be introduced into the DCs in order to elicit a host immune response. In our view however, caTLR4 is the most potent activating molecule and is therefore preferred.

The term “target” used throughout the description is not limited to the specific examples that may be described herein. Any infectious agent such as a virus, a bacterium or a fungus may be targeted.

The term “target-specific antigen” used throughout the description is not limited to the specific examples that may be described herein. It will be clear to the skilled person that the invention is related to the induction of immunostimulation in APCs, regardless of the target-specific antigen that is presented. The antigen that is to be presented will depend on the type of target to which one intends to elicit an immune response in a subject. Typical examples of target-specific antigens are expressed or secreted markers that are specific to bacterial and fungal cells or to specific viral proteins or viral structures.

Target-specific antigens are preferably selected from region in the pathogenic genome which are rather stable, i.e. wherein little variation between different strains of the same pathogenic species are observed. For short-term solutions, i.e. the development of vaccines for subjects which are already infected are at high risk to become infected, the best target antigens are likely the “M” (matrix) and/or “N” (nucleocapsid) proteins and the non-structural proteins. For a ring-fence emergency vaccine, intended to be used to prevent spreading in high risk areas and close contact individuals an interesting combination is an mRNA vaccine containing S (spike) and M/N targets, delivered intranasally. For long-term solutions, such as preventive vaccination, the best solution is a “universal” vaccine that can be rapidly deployed at a next incident. The high variability of the spike protein, the different receptors used, and the doubts on neutralizing potential makes a universal antibody-based vaccine unlikely. A T cell based vaccine against conserved regions across major pathogenic strains is in that instance much more feasible. In one particular embodiment, an artificially constructed immunogen consisting of strong T cell stimulatory epitopes from the pathogen's genome, and removing any T suppressing epitopes would confer such strong and broad protection. Alternatively the antigen may be designed such as to induce an antibody response in a subject.

The term “infectious disease” or “infection” used throughout the description is not intended to be limited to the types of infections that may have been exemplified herein. The term therefore encompasses all infectious agents to which vaccination would be beneficial to the subject. Non-limiting examples are the following virus-caused infections or disorders: Acquired Immunodeficiency Syndrome—Adenoviridae Infections—Alphavirus Infections—Arbovirus Infections—Bell Palsy—Borna Disease—Bunyaviridae Infections—Caliciviridae Infections—Chickenpox—Common Cold—Condyloma Acuminata—Coronaviridae Infections—Coxsackievirus Infections—Cytomegalovirus Infections—Dengue—DNA Virus Infections—Contagious Ecthyma,—Encephalitis—Encephalitis, Arbovirus—Encephalitis, Herpes Simplex—Epstein—Barr Virus Infections—Erythema Infectiosum—Exanthema Subitum—Fatigue Syndrome, Chronic—Hantavirus Infections—Hemorrhagic Fevers, Viral—Hepatitis, Viral, Human—Herpes Labialis—Herpes Simplex—Herpes Zoster—Herpes Zoster Oticus—Herpesviridae Infections—HIV Infections—Infectious Mononucleosis—Influenza in Birds—Influenza, Human—Lassa Fever—Measles—Meningitis, Viral—Molluscum Contagiosum—Monkeypox—Mumps—Myelitis—Papillomavirus Infections—Paramyxoviridae Infections—Phlebotomus Fever—Poliomyelitis—Polyomavirus Infections—Postpoliomyelitis Syndrome—Rabies—Respiratory Syncytial Virus Infections—Rift Valley Fever—RNA Virus Infections—Rubella—Severe Acute Respiratory Syndrome—Slow Virus Diseases—Smallpox—Subacute Sclerosing Panencephalitis—Tick—Borne Diseases—Tumor Virus Infections—Warts—West Nile Fever—Virus Diseases—Yellow Fever—Zoonoses—Etc. Specific antigens for viruses can be HIV-gag, -tat, -rev or -nef, or Hepatitis C-antigens; particularly preferred virus-caused infections or disorders are Coronaviridae Infections, such as infections caused by coronavirus 229E, coronavirus OC43, SARS-CoV, HCoV NL63, HKU1, MERS-CoV or COVID-19.

Further non-limiting examples are the following bacteria- or fungus-caused infections or disorders: Abscess—Actinomycosis—Anaplasmosis—Anthrax—Arthritis, Reactive—Aspergillosis—Bacteremia—Bacterial Infections and Mycoses—Bartonella Infections—Botulism—Brain Abscess—Brucellosis—Burkholderia Infections—Campylobacter Infections—Candidiasis—Candidiasis, Vulvovaginal—Cat—Scratch Disease—Cellulitis—Central Nervous System Infections—Chancroid—Chlamydia Infections—Chlamydiaceae Infections—Cholera—Clostridium Infections—Coccidioidomycosis—Corneal Ulcer—Cross Infection—Cryptococcosis—Dermatomycoses—Diphtheria—Ehrlichiosis—Empyema, Pleural—Endocarditis, Bacterial—Endophthalmitis—Enterocolitis, Pseudomembranous—Erysipelas—Escherichia coli Infections—Fasciitis, Necrotizing—Fournier Gangrene—Furunculosis—Fusobacterium Infections—Gas Gangrene—Gonorrhea—Gram—Negative Bacterial Infections—Gram—Positive Bacterial Infections—Granuloma Inguinale—Hidradenitis Suppurativa—Histoplasmosis—Hordeolum—Impetigo—Klebsiella Infections—Legionellosis—Leprosy—Leptospirosis—Listeria Infections—Ludwig's Angina—Lung Abscess—Lyme Disease—Lymphogranuloma Venereum—Maduromycosis—Melioidosis—Meningitis, Bacterial—Mycobacterium Infections—Mycoplasma Infections—Mycoses—Nocardia Infections—Onychomycosis—Osteomyelitis—Paronychia—Pelvic Inflammatory Disease—Plague—Pneumococcal Infections—Pseudomonas Infections—Psittacosis—Puerperal Infection—Q Fever—Rat—Bite Fever—Relapsing Fever—Respiratory Tract Infections—Retropharyngeal Abscess—Rheumatic Fever—Rhinoscleroma—Rickettsia Infections—Rocky Mountain Spotted Fever—Salmonella Infections—Scarlet Fever—Scrub Typhus—Sepsis—Sexually Transmitted Diseases, Bacterial—Sexually Transmitted Diseases, Bacterial—Shock, Septic—Skin Diseases, Bacterial—Skin Diseases, Infectious—Staphylococcal Infections—Streptococcal Infections—Syphilis—Syphilis, Congenital—Tetanus—Tick—Borne Diseases—Tinea—Tinea Versicolor—Trachoma—Tuberculosis—Tuberculosis, Spinal—Tularemia—Typhoid Fever—Typhus, Epidemic Louse—Borne—Urinary Tract Infections—Whipple Disease—Whooping Cough—Vibrio Infections—Yaws—Yersinia Infections—Zoonoses—Zygomycosis—Etc.

In a preferred embodiment of the vaccine of the invention, the mRNA or DNA molecule(s) encode(s) the CD40L and CD70 immunostimulatory proteins. In a particularly preferred embodiment of the vaccine of the invention, the mRNA or DNA molecule(s) encode(s) CD40L, CD70, and caTLR4 immunostimulatory proteins.

Said mRNA or DNA molecules encoding the immunostimulatory proteins can be part of a single mRNA or DNA molecule. Preferably, said single mRNA or DNA molecule is capable of expressing the two or more proteins simultaneously. In a further embodiment, the two or more mRNA or DNA molecules encoding the immunostimulatory proteins are part of a single mRNA or DNA molecule. This single mRNA or DNA molecule is preferably capable of expressing the two or more proteins independently. In a preferred embodiment, the two or more mRNA or DNA molecules encoding the immunostimulatory proteins are linked in the single mRNA or DNA molecule by an internal ribosomal entry site (IRES), enabling separate translation of each of the two or more mRNA sequences into an amino acid sequence. Alternatively, a selfcleaving 2a peptide-encoding sequence is incorporated between the coding sequences of the different immunostimulatory factors. This way, two or more factors can be encoded by one single mRNA or DNA molecule. Preliminary data where cells were electroporated with mRNA encoding CD40L and CD70 linked by an IRES sequence or a self cleaving 2a peptide shows that this approach is indeed feasible.

The invention thus further provides for an mRNA molecule encoding two or more immunostimulatory factors, wherein the two or more immunostimulatory factors are either translated separately from the single mRNA molecule through the use of an IRES between the two or more coding sequences. Alternatively, the invention provides an mRNA molecule encoding two or more immunostimulatory factors separated by a selfcleaving 2a peptide-encoding sequence, enabling the cleavage of the two protein sequences after translation.

In any embodiment, said target-specific antigen is selected from the group consisting of: total mRNA isolated from (a) target cell(s), one or more specific target mRNA molecules, protein lysates of (a) target cell(s), specific proteins from (a) target cell(s), a synthetic target-specific peptide or protein and synthetic mRNA or DNA encoding a target-specific antigen or its derived peptide(s). Said target can be viral, bacterial or fungal, proteins or mRNA, in particular mRNA molecules designed for induction of antibody responses.

The mRNA or DNA used or mentioned herein can either be naked mRNA or DNA, or protected mRNA or DNA. Protection of DNA or mRNA increases its stability, yet preserving the ability to use the mRNA or DNA for vaccination purposes. Non-limiting examples of protection of both mRNA and DNA can be: liposome-encapsulation, protamine-protection, (Cationic) Lipid Lipoplexation, lipidic, cationic or polycationic compositions, Mannosylated Lipoplexation, Bubble Liposomation, Polyethylenimine (PEI) protection, liposome-loaded microbubble protection, lipid nanoparticles, etc.

In some preferred embodiments, the mRNA used in the methods of the present invention has a 5′ cap structure with a so-called CAP-1 structure, meaning that the 2′ hydroxyl of the ribose in the penultimate nucleotide with respect to the cap nucleotide is methylated.

In another particular embodiment said mRNA molecule is a self-amplifying or trans-amplifying mRNA molecule. Self-amplifying mRNA molecules typically encode the antigen as well as a viral replication machinery that enables intracellular RNA amplification and abundant protein expression. Trans-amplifying mRNA molecules use a similar principle although the antigen and viral replication machinery are encoded from different mRNA molecules.

In another particular embodiment, two, three, four, . . . or all of the used mRNA molecules of the present invention have a 5′ cap structure with a so-called CAP-1 structure.

In a further embodiment, one or more of the mRNA molecules of the present invention may further comprise at least one modified nucleoside. In another particular embodiment, two, three, four, . . . or all of the used mRNA molecules of the present invention have at least one modified nucleoside.

In another particular embodiment of the present invention, said mRNA molecules further comprise at least one modified nucleoside, such as selected from the list comprising pseudouridine, 5-methoxy-uridine, 5-methyl-cytidine, 2-thio-uridine, and N6-methyladenosine.

In a particular embodiment of the present invention, said at least one modified nucleoside may be a pseudouridine, such as selected from the list 4-thio-pseudouridine, 2-thio-pseudouridine, 1-carboxymethyl-pseudouridine, 1-propynyl-pseudouridine, 1-taurinomethyl-pseudouridine, N1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydropseudouridine, 2-thio-dihydropseudouridine, 4-methoxy-pseudouridine, and 4-methoxy-2-thio-pseudouridine. In a very specific embodiment, said at least one modified nucleoside is N1-methyl-pseudouridine.

Alternative nucleoside modifications which are suitable for use within the context of the invention, include: pyridin-4-one ribonucleoside, 5-aza-uridine, 2-thio-5-aza-uridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, I-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the mRNA comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the mRNA comprises at least one nucleoside selected from the group consisting of 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In some embodiments, mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guaiguanosine, and N2,N2-dimethyl-6-thio-guanosine.

The mRNA molecules used in the present invention may contain one or more modified nucleotides, in particular embodiment, at least 10%, 20%, 30%, 40%, 50%, 60%, 70%, 80%, 90% or 100% of a particular type of nucleotides may be replaced by a modified one. It is also not excluded that different nucleotide modifications are included within the same mRNA molecule. In a very specific embodiment of the present invention, about 100% of uridines in said mRNA molecules is replaced by N1-methyl-pseudouridine.

In a specific embodiment, one or more of said mRNA molecules of the present invention may further contain a translation enhancer and/or a nuclear retention element. Suitable translation enhancers and nuclear retention elements are those described in WO2015071295.

The combinations and vaccines of the present invention are particularly formulated for intranasal administration.

In the context of the present invention, the term “nasal administration” or “intranasal administration” is meant to be a route of administration in which the compositions/vaccines of the present invention are applied in the nasal cavity. The nasal mucosa can be used for non-invasive topical or systemic administration of components. More specifically in the context of the present invention, using such intranasal administration forms, the mRNA molecules of the present invention may be brought into direct contact with antigen presenting cell in the upper respiratory tract and induce several protective T cells like resident memory CD8+ T cells, thereby inducing local immunity against respiratory tract infections. This also reduces the risk of pathogen spreading to the lower respiratory tract, and also reduces disease pathology.

Any formulation allowing such intranasal administration is suitable for use within the context of the present invention. In particular, some specific, non-limiting examples are provided herein below:

In a very easy set-up, the compositions/vaccines of the present invention may be administered by simply injecting a therapeutically acceptable solution comprising one or more of the mRNA molecules in the oronasopharangeal cavity, such as in the format of a dropper. Alternatively, unit/bidose systems may be used, specifically where administration requires exact dosing. These systems contain one or two separated half doses ready for administration.

Therapeutically acceptable solutions for intranasal administration are preferably selected such that they do not impact the stability of the mRNA encompassed therein. Moreover, such solutions preferably increase RNA uptake in antigen presenting cells of the oronasopharangeal cavity. Accordingly, classical RNA transfection buffers/components may be used, such as jetPEI®, Lipofectamine®, RiboJuice® or Stemfect®.

The jetPEI® tranfection agent is a linear polyethyleneimine derivative (in particular a polyplex). Accordingly in a specific embodiment, the intranasal administration may be performed in the presence of polyethyleneimine and/or derivatives thereof.

Lipofectamine consists of a 3:1 mixture of DOSPA (2,3-dioleoyloxy-N-[2(sperminecarboxamido) ethyl]-N,N-dimethyl-1-propaniminium trifluoroacetate) and DOPE (1,2-Dioleoyl-sn-glycero-3-phosphoethanolamine).

Alternatively, the compositions/vaccines of the present invention may be formulated in the form of an aerosol spray, nasal spray, multi-dose spray pump, . . . . In a multi-dose spray pump, the compositions/vaccines may be filled into bottles made of glass or plastic materials, which are closed by attaching the nasal spray pump including a dip tube. Nasal spray pumps are displacement pumps and when actuating the pump by pressing the actuator towards the bottle, a piston moves downward in the metering chamber. A valve mechanism at the bottom of the metering chamber will prevent backflow into the dip tube. So, the downward movement of the piston will create pressure within the metering chamber which forces the air or the liquid outwards through the actuator and generates the spray. When the actuation pressure is removed, a spring will force the piston and actuator to return to its initial position. This creates and underpressure in the metering chamber which pulls the liquid from the container by lifting up the ball from the ball seat above the dip tube at the bottom of the metering chamber. The metering chamber ensures the right dosing and an open swirling chamber in the tip of the actuator will aerosolize the metered dose.

For most nasal spray pumps the dispensed volume pre actuation is set between 50 and 150 μl, and an administered volume of about 100 μl per nostril is optimum for adults, since higher volumes are prone to drip out. So the anticipated dose is preferably fit into a volume of roughly 100-200 μl when both nostrils are spayed.

Depending on the intended purpose, the intranasal composition may be administered according to a particular administration scheme, such as once, twice or thrice daily.

Alternatively, the intranasal administration may be administered every two, three, four, five, six or seven days, such as once per week or alternatively once per 2 weeks. For each of said administrations, the dosing may also be varied, such as a higher dose at the beginning of the treatment, and a lower dose towards the end of the treatment. The protocol of use contains specific instruction to minimize uptake by the lungs, such a holding breath or breathing out after the administration.

The compositions of the present invention may be used as a prophylactic composition (such as prior to the manifestation of symptoms) or alternatively as a therapeutic composition (such as when symptoms have already emerged).

Given the unstable nature of mRNA molecules, these are preferably in a protected format such as defined herein above; more specifically, they may be included in for example lipid nanoparticles. Hence, the present invention also provides a combination or composition as defined herein; wherein one or more of said mRNA molecules are encompassed in nanoparticles; such as lipid-based nanoparticles or polyplexes, lipoplexes and polylipoplexes.

As used herein, the term “nanoparticle” refers to any particle having a diameter making the particle suitable for systemic, in particular intravenous administration, of, in particular, nucleic acids, typically having a diameter of less than 1000 nanometers (nm).

In a specific embodiment of the present invention, the nanoparticles are selected from the list comprising: lipid nanoparticles and polymeric nanoparticles.

A lipid nanoparticle (LNP) is generally known as a nanosized particle composed of a combination of different lipids. While many different types of lipids may be included in such LNP, the LNP's of the present invention may for example be composed of a combination of an ionisable lipid, a phospholipid, a sterol and a PEG lipid.

A polymeric nanoparticle can typically be a nanosphere or a nanocapsule. Two main strategies are used for the preparation of polymeric nanoparticles, i.e. the “top-down” approach and the “bottom-up” approach. In the top-down approach, a dispersion of preformed polymers produces polymeric nanoparticles, whereas in the bottom-up approach, polymerization of monomers leads to the formation of polymeric nanoparticles. Both top-down and bottom-up methods use synthetic polymers/monomers like poly(d, l-lactide-co-glycolide), poly(ethyl cyanoacrylate), poly(butyl cyanoacrylate), poly(isobutyl cyanoacrylate), and poly(isohexyl cyanoacrylate); stabilizers like poly(vinyl alcohol) and didecyldimethylammonium bromide; and organic solvents like dichloromethane and ethyl acetate, benzyl alcohol, cyclohexane, acetonitrile, acetone, and so on. Recently the scientific community has been trying to find alternatives for synthetic polymers by using natural polymers and synthesis methods with less toxic solvents.

The present invention also provides the combinations and vaccines as defined herein for use in human or veterinary medicine, in particular for use in the treatment of pathogenic infections, more in particular, respiratory infections, such as viral infections.

Finally, the present invention provides a method for the treatment of a pathogenic infections comprising the steps of administering to a subject in need thereof a combination or vaccine of the present invention.

The compositions may also be of value in the veterinary field, which for the purposes herein not only includes the prevention and/or treatment of diseases in animals, but also—for economically important animals such as cattle, pigs, sheep, chicken, fish, etc.—enhancing the growth and/or weight of the animal and/or the amount and/or the quality of the meat or other products obtained from the animal.

The subject to be treated is preferably suffering from a disease or disorder selected from the group comprising: bacterial, viral or fungal infection.

As used herein the term ‘prevention’ is meant to be reducing the risk of being infected or reducing the symptoms associated with a pathogenic infection.

### EXAMPLES

### Example 1: Short Term Crisis

In the (unlikely) scenario that a crisis really derails into a world-wide pandemic, the threshold for emergency product will go down fast. Referring to the (imminent) Flu pandemic, several vaccines with totally new adjuvant technology got the chance to be rapidly tested in that setting.

In such event, any of the following options can be followed:

A) A “killer” T cell based vaccine—to be used in high risk for contamination or infected individuals. The best targets are likely the “M” (matrix) and/or “N” (nucleocapsid) proteins.

B) A ring-fence emergency vaccine—to be used to prevent spreading in high risk areas and close contact individuals. In such instance, an interesting combination is likely an mRNA vaccine containing S (spike) and M/N targets, delivered intranasally. A surprisingly good result is obtained (Phua, Leong, & Nair, 2013; Phua, Staats, Leong, & Nair, 2014) in mice tumor models with an intranasal delivery protocol adapted by the researchers from the Stemfect® transfection kit from Stemgent.

### Example 2: Long Term Solution

Mass preventive vaccination against all possible corona or other types of pathogens seems unlikely. Not only because of the variability of the strains, but also because the unpredictability of timing and place of strike, and so the impossible task to define who is at risk.

The best solution is thus a “universal” vaccine that can be rapidly deployed at a next incident. The high variability of the spike protein, the different receptors used, and the doubts in a broadly-neutralizing potential make a universal antibody-based vaccine unlikely. A T cell based vaccine against conserved regions across major pathogenic strains seems much more feasible. A thorough analysis of genetic make-up of the pathogenic family and a smart design using epitope prediction and fusion constructs gives the best possible candidate.

### Example 3: Development Outline

Step 1: Exploratory Mouse Experiments (Biodistribution, Concept and Safety):
Intranasal Fluc biodistribution study
Trimix—model antigen (eg E7)—intranasal—immune read-outs and nose/airways histopathology.

Research grade production of M, N and S mRNA, as well as mRNA encoding structural and non-structural proteins

Fast-track scientific advice: Innovation office, sFDA

Step 2: Mouse Enabling Immuno and Tox:
Trimix—M/S—intranasal—nCoV immune read-outs and full tox histopath.

Adapt StemFect as required and supply (at minimum GMP-like quality)

Research grade production for challenge study—M* and S*

GMP grade production of M/S mRNA (and Trimix)

Clinical trial submission

Step 3: Phase I into II Trial in Healthy Volunteers:
schedule 0d (optionally 7d). 25 subjects in phase I
step up to min 250 subjects.
Safety parameters and immune read-outs (include IgA if S is used).

Animal challenge model:
Trimix—M* (+S*)—intranasal—immunized day 0 (optionally day 7) challenge with species specific corona strain—establish protection/immune correlates (50 animals)

Commercial manufacturing and consistency

Emergency use file submission

### Example 4: Preclinical Product Development Approach

The preclinical program consists of 4 steps:
1. A respiratory tract expression and distribution assessment. Using the unique possibility of monitoring expression of FLUC mRNA in vivo by bioluminescence a first experiment in mice evaluates our 2 to 3 potential nasal delivery systems (naked mRNA, StemFect and in-house LNP) and confirm delivery and expression in the nasal cavity and absence of expression or low expression in the lung. The delivery system is selected based on its performance in this assay and its general manufacturability properties.
2. The induction of T cell immune responses is assessed in a second mouse experiment. Here 2 model antigens for which we have in-house and published experience and immunological tools are administered under 2 dosing regimens (0, 8 days and 0,−22 days). A full evaluation of all T cell compartments (mucosal, lung, lymph nodes, systemic) as well as a safety evaluation of respiratory tract and selected organs confirms the immunological hypothesis and the expected safety profile of the platform.
3. A GLP repeated dose tox study enables the progression to clinical use of the vaccine. Based on our previous experience with mRNA vaccine we prefer to select a single species. This study allows to confirm the induction of relevant immune response by the COVID-19 target, according to the responses predicted during vaccine design. Toxicity evaluation has already been performed for TriMix+antigen mRNA for parenteral administration. The key focus of this evaluation is on the delivery system. Additionally, supporting genotoxicity and pharmacological studies are added to the plan for selected constituents of the delivery system. Depending on the findings in experiment 1 a special attention will be needed towards secondary effects in the lung. Some additional studies could be run in parallel to start of phase I.
4. A challenge and disease prevention study in animals. This step is proposed in parallel with the clinical study. The selection of the relevant animal species and viral strain is subject to collaboration within the network of contributors to the corona vaccine effort. This study shows that the vaccine prevents development of lower respiratory disease in animals vaccinated with our product and challenged after immunization with virus. Immune assessment allows to correlate this protection to immune response, that can then in turn be compared to the responses observed in human subjects. The use of an animal challenge allows to explore the potential of the vaccine to generate a broad response protecting against strain drifts or new corona family members.

### Example 5: Clinical Development Approach

Our approach for clinical development is to focus on safety and immunogenicity—and draw a correlation to an animal challenge model to support expected efficacy. To cater towards the use as an emergency vaccine a fluid transition from phase I into II allows for the fastest generation of the required data. For the same reason we choose a short induction schedule. Contrary to the induction of humoral response such short administration schedule does lead to good results in T cell immunity.

The safety of the product is assessed in 3 steps:
1. Measuring T cell immunity on nasal mucosa is a relative new approach and has been published only in a few papers. Nasal samples from vaccinated individuals are collected longitudinally using minimally-invasive curettage as described previously (Jochems et al., 2018 & 2019). Established cryopreservation protocols allow for batch analysis. This allows us to measure in parallel i) in vivo responses to vaccination by phenotyping and ii) antigen-specific responses. In vivo T cell, including tissue-resident memory T cells, B cell and DC responses are characterized in depth using mass cytometry with panels targeted at the nasal immune system. This assay is now miniaturized at LUMC, to analyze nasal curettage samples. The establishment of antigen-specific immunity in the nose is assessed by co-culturing nasal cells with vaccine activated monocyte-derived dendritic cells from PBMC from the same individual. In-house protocols are adapted to be able to assess mucosal responses. Cytokine production (IFNγ, TNFα, etc.) are measured in supernatant, while CD40L and CTLA-4 induction on T cells is phenotyped by flow cytometry to measure antigen-specific stimulation. The concurrent characterization of cellular phenotype and functionality using longitudinal minimally-invasive samples collected from the human nasal mucosa holds significant potential to rapidly predict vaccine success. Adaptation of these methods to our particular protocol is performed in parallel with the preclinical phase of the program.
2. A phase I multiple ascending dose study in healthy human volunteers. Based on the preclinical results, we select the starting and targeted dose/schedule for this study. The first part of the study is a rapid step-up (eg 3 subjects per step) from starting to target regimen primarily evaluating safety. The endpoints for this study are safety (clinical evaluation, patient reporting and blood analysis), systemic (PBMC) and mucosal (nasal sampling) immunity assessment. Around 40 subjects are included in this study, of which minimum 25 are dosed with the target dose/schedule. Nasal samples are collected prior to vaccination (days −5 and −1), early following vaccination (days 3 and 7) and for longer time follow-up (weeks 2, 3, 4 and 8).
3. This initial phase I is followed by an extension, also in healthy volunteers, into a phase II immunogenicity study. With an expanded number of subjects included (n=100) with the selected vaccine schedule it allows for a robust assessment of the induced immune response, its variability, its longer term dynamics and its correlation to the animal models and protection. Continuing the study within the same setup and network offers obvious advantages towards consistency and speed of data generation.

### Example 6: In Vivo Intranasal Administration in Mice

Material and Methods

Mice

A total of 48 mice (Mus musculus) were obtained from Charles River and acclimated for at 14 days prior to study initiation. During acclimation, animals were assigned to a group based on weight and identified by tail tattoo.

Construct Design

The full length coding sequence of influenza NP protein (Influenza A/NL/18/94 H3N2) was cloned in frame to signal sequence and DC lamp sequence in order to optimize processing and presentation in MHC complexes. To improve expression and reduce immunogenic response towards the mRNA construct N1 methyl pseudouridine modifications were used.

In combination with the TriMix mRNAs, the immunogenic construct was used at a fixed 1:1 ratio.

Administrations

On Day 0, Day 7, Day 14, candidate and control administrations were performed intranasally according to group attribution as detailed below (16 mice per group)

On Day 42, all animals from NP/TriMix mod (in vivo jetPEI) (Group 1), NP-mod (in vivo jetPEI) (group 2) or PBS (Group 3) were challenged intranasally (1 LD50, 10 μl).

On terminal time point (Day 48), animals received CD45.2-BV605 antibody (Biolegend, Clone 104, 3 μg) injected intravenously 5 minutes before euthanasia. Lung were collected and lung-infiltrating immune (left lobe) were used for viral titration.

Immunization on Day 0, Day 7 and Day 14:

On Day 0, all animals were administered intranasally with 30 μL (15 μL per nostril) of candidate preparations (Groups 1 and 2) or PBS (Group 3) with a micropipette. Thirty microliters (30 μL) (15 μL per nostril) of candidate preparations (Groups 1 and 2) or PBS (Group 3) were administered intranasally with a micropipette on Day 7 and Day 14. Animals were administered under anaesthesia.

Intranasal Immunization (15 μL per nostril) was performed with 3.75 μg/3.75 μg of NP/TriMix mod (in vivo jetPEI) (Group 1) or 7.5 μg of NP-mod (in vivo jetPEI) (Group 2) on Day 0, Day 7 and Day 14.

Viral Infection:

All animals from groups treated with NP/TriMix mod (in vivo jetPEI) (Group 1), NP-mod (in vivo jetPEI) (Group 2) or vehicle (Group 3) were challenged with influenza A PR8 intranasally (1 LD50, 10 μl) on Day 42.

Lung Isolation:

On terminal time (Day 48), animals were euthanized by carbon dioxide asphyxiation and gross necropsy was performed prior to organ collection.

The lung (left lobe) were collected aseptically, weighted and placed in 0.5 mL of collection medium ((49% DMEM (Gibco, Cat. 11965-084) and 49% Medium 199 (Gibco, Cat. 11150-059), supplemented with 0.1% of FBS (Gibco, Cat. 26140-079)) in a Precellys tube at 4° C. Lung in Precellys tubes were homogenized, aliquoted and frozen for viral titration.

Influenza Viral Load Estimations in Lung Tissue Samples: (TCID50)

Lung samples collected for viral load estimation (Day 48) were disrupted with two 20-seconds cycles at 5000 rpm with a 5-seconds pause between cycles. Tissue homogenates were vortexed for several seconds before and after 0.5 ml of DMEM/Medium-199, 0.1% FBS was added to the tube. Tissue homogenates were cleared of tissue fragments with a 10 minutes centrifugation at 3200×g and 4° C. Cleared supernatants were collected and aliquoted and frozen for viral titration.

Lung samples were filter-sterilized (5 minutes at 14000×g and 4° C.), using Spin-X tubes (Corning, Cat. 8160). Ten-fold dilutions of the filtered lung samples were made in titration medium ((49% DMEM (Gibco, Cat. 11965-084) and 49% Medium 199 (Gibco, Cat. 11150-059), supplemented with 0.1% of FBS (Gibco, Cat. 26140-079), 1× GlutaMax (Gibco, Cat. 35050-061) and 0.1% Gentamicin (Gibco, Cat. 15750-060)), with a starting dilution of ½, in sterile microtiter polypropylene tubes. MDCK cells were trypsinized, pooled and resuspended at 2.4×105 cells/mL in titration medium. 50 μL of sample serial-dilutions were added to the appropriate wells (octuplicates) of 96-well plates and 2.4×104 MDCK cells (100 μL) were added to all wells. Samples, in a total volume of 200 μL, were incubated for 7 days at 37° C. and 5% CO2 to allow viral replication.

TCID50 was evaluated by hemagglutination, which was achieved by mixing 50 μL of viral supernatants with 50 μL of 0.5% chicken red blood cells in V-bottom 96-well plates. Plates were incubated 1 hour at RT and hemagglutination was read.

Results

Viral load estimation in lung samples:

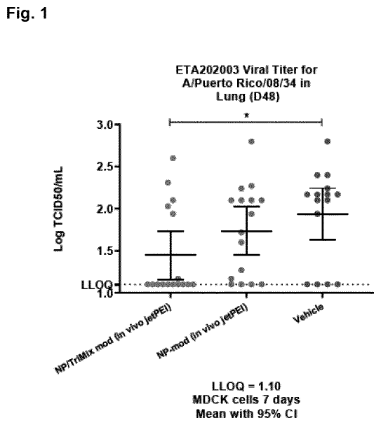

Influenza virus quantitation by TCID50 in lungs showed 10 out of 16 animals in NP/Trimix mod (in vivo jetPEI) (Group 1) had a viral titer below limit of quantification with only 4 animals for groups treated with NP mod (in vivo jetPEI) (Group 2) and left untreated (Group 3) (FIG. 1).

Accordingly, the compositions of the present invention are capable of reducing viral loads in challenged mice when administered intranasally.

### REFERENCES

Jochems S P, de Ruiter K, Solórzano C, Voskamp A, Mitsi E, Nikolaou E, Carniel B F, Pojar S, German E L, Reiné J, Soares-Schanoski A, Hill H, Robinson R, Hyder-Wright A D, Weight C M, Durrenberger P F, Heyderman R S, Gordon S B, Smits H H, Urban B C, Rylance J, Collins A M, Wilkie M D, Lazarova L, Leong S C, Yazdanbakhsh M, Ferreira D M. Innate and adaptive nasal mucosal immune responses following experimental human pneumococcal colonization. J Clin Invest. 2019 Jul. 30; 130:4523-4538
Jochems S P, Marcon F, Carniel B F, Holloway M, Mitsi E, Smith E, Gritzfeld J F, Solórzano C, Reiné J, Pojar S, Nikolaou E, German E L, Hyder-Wright A, Hill H, Hales C, de Steenhuijsen Piters W A A, Bogaert D, Adler H, Zaidi S, Connor V, Gordon S B, Rylance J, Nakaya H I, Ferreira D M. Inflammation induced by influenza virus impairs human innate immune control of pneumococcus. Nat Immunol. 2018 December; 19(12):1299-1308
Phua, K. K. L., Leong, K. W., & Nair, S. K. (2013). Transfection efficiency and transgene expression kinetics of mRNA delivered in naked and nanoparticle format. Journal of Controlled Release, 166(3), 227-233. https://doi.org/10.1016/j.jconrel.2012.12.029
Phua, K. K. L., Staats, H. F., Leong, K. W., & Nair, S. K. (2014). Intranasal mRNA nanoparticle vaccination induces prophylactic and therapeutic anti-tumor immunity. Scientific Reports, 4, 4-10. https://doi.org/10.1038/srep05128
Roper, R. L., & Rehm, K. E. (2009). SARS vaccines: Where are we? Expert Review of Vaccines, 8(7), 887-898. https://doi.org/10.1586/erv.09.43

1. A combination comprising:
one or more mRNA molecules encoding for a functional immunostimulatory protein selected from the list comprising CD40L, caTLR4 and CD70; and
one or more mRNA molecules encoding a bacterial, viral or fungal antigen;
wherein said combination is in the form of an intranasal formulation.

2. The combination of claim 1, wherein said one or more mRNA molecules encode for all of said functional immunostimulatory proteins CD40L, caTLR4 and CD70.

3. The combination as defined in anyone of claim 1 or 2; wherein said antigen is an antigen from a respiratory tract pathogen.

4. The combination as defined in anyone of claims 1 to 3; wherein said antigen is an M (matrix), N (nucleocapid) or S (spike) antigen, an artificial antigen designed to contains T cell stimulatory epitopes and suppress T regulatory epitopes or a surface antigen designed to elicit antibody responses.

5. The combination as defined in claim 3; wherein said respiratory tract pathogen is a coronavirus.

6. The combination as defined in anyone of claims 1 to 5, wherein said mRNA molecules are formulated in the form of nanoparticles, such as lipid-based nanoparticles.

7. The combination as defined in anyone of claims 1 to 5; wherein said mRNA molecules are formulated in the form of lipoplexes, dendrimers, polyplexes or hybrid lipopolyplexes.

8. The combination as defined in claim 7, wherein said mRNA molecules are formulated in the form of a polyplex using polyethylenimine.

9. The combination as defined in anyone of claims 1 to 8, wherein one or more of said mRNA molecules comprise a 5′ CAP-1 structure.

10. The combination as defined in anyone of claims 1 to 9; wherein one or more of said mRNA molecules comprise one or more modified nucleosides, in particular N1-methyl-pseudouridine.

11. A vaccine comprising the combination of any one of claims 1 to 10.

12. The combination as defined in any one of claims 1 to 10 or the vaccine as defined in claim 11 for use in human or veterinary medicine.

13. The combination as defined in any one of claims 1 to 10, or the vaccine as defined in claim 11 for use in the prevention and/or treatment of an infectious disease.

14. A method for the prevention or treatment of an infectious disease, said method comprising administering to a subject in need thereof a combination as defined in anyone of claims 1 to 10 or a vaccine as defined in claim 11.
